(19) United States
(12) Patent Application Publication
(10) Pub. No.: US 2016/0138114 A1
(43) Pub. Date: May 19, 2016

(54) Biomarkers of Response to Inhibition of Poly-ADP Ribose Polymerase (PARP) in Cancer

(71) Applicant: THE REGENTS OF THE UNIVERSITY OF CALIFORNIA, Oakland, CA (US)

(72) Inventors: Dennis Slamon, Woodland Hills, CA (US); Richard Finn, Encino, CA (US); Dylan Conklin, Woodland Hills, CA (US)

(73) Assignee: THE REGENTS OF THE UNIVERSITY OF CALIFORNIA, Oakland, CA (US)

(21) Appl. No.: 14/899,974

(22) PCT Filed: Jun. 18, 2014
(86) PCT No.: PCT/US14/42993

§ 371 Date: Dec. 18, 2015

Related U.S. Application Data
(60) Provisional application No. 61/836,987, filed on Jun. 19, 2013.

(51) Int. Cl. C12Q 1/68; G01N 33/574; A61K 45/06; A61K 31/5025

(52) U.S. Cl. CPC C12Q 1/6886; A61K 31/5025; G01N 33/57496; A61K 45/06; C12Q 2600/158; C12Q 2600/106; G01N 2333/914

(57) ABSTRACT

Provided herein are methods of identifying a subject having a poly-ADP ribose polymerase (PARP) inhibitor-sensitive tumor by detecting a genomic gain in chromosome 1q21 and/or chromosome 20q13.3 in a tumor sample from the subject. Also provided are methods of identifying a subject having a PARP inhibitor-sensitive tumor by detecting gene amplification of a CHD1L gene or an RTEL1 gene in a tumor sample from the subject. Further provided are methods of treating a tumor with a genomic gain in chromosome 1q21 and/or chromosome 20q13.3 in a subject by administering an effective dose of a PARP inhibitor.

### CROSS-REFERENCE TO RELATED APPLICATION

This application claims the benefit of priority under 35 U.S.C. §119(e) of U.S. Ser. No. 61/836,987, filed Jun. 19, 2013, the entire content of which is incorporated herein by reference.

### BACKGROUND OF THE INVENTION

1. Field of the Invention

The invention relates generally to the use of biomarkers for the detection, diagnosis, and treatment of cancer, and more specifically to the identification of tumor cells that are susceptible to treatment with poly-ADP ribose polymerase (PARP) inhibitors.

2. Background Information

Poly(ADP-ribose) polymerase (PARP) is a nuclear enzyme that has been implicated in several biological processes, including DNA repair, gene transcription, cell cycle progression (including proliferation and differentiation), cell death, chromatin functions, genomic (e.g., chromosomal) stability and telomere length. PARP's main function is to detect single-strand DNA breaks and to signal such breaks to the enzymatic machinery involved in the DNA break repair.

Activation of PARP can be induced by DNA strand breaks after exposure to chemotherapy, ionizing radiation, oxygen free radicals, or nitric oxide (NO). Once PARP detects a DNA break, it binds to the DNA, and synthesizes a poly (ADP-ribose) chain (PAR) as a signal for the other DNA-repairing enzymes. Because this cellular ADP-ribose transfer process is associated with the repair of DNA strand breakage in response to DNA damage caused by radiotherapy or chemotherapy, it can contribute to the resistance that often develops to various types of cancer therapies. Enhanced PARP-1 expression and/or activity may allow tumor cells to withstand genotoxic stress and increase their resistance to DNA-damaging agents. As a consequence, inhibition of PARP-1 through small molecules has been shown to sensitize tumor cells to cytotoxic therapy (e.g. temozolomide, platinums, topoisomerase inhibitors and radiation). In addition, PARP inhibitors have been used as monotherapy in some cancers.

However, there is currently no reliable method or biomarker for consistently identifying patients with cancers that are likely to respond to PARP inhibitor therapies. Such a biomarker as it would allow for appropriate selection of patients who may benefit from this treatment approach.

### SUMMARY OF THE INVENTION

The present invention is based on the finding that genomic gains in chromosomes 1q21 and 20q13.3 are strongly associated with predicting response of tumor cells to PARP inhibitors. Within these chromosomal regions, amplification of the gene CHD1L (also designated ALC1) located at chromosome 1q21 and/or the gene RTEL1 located at chromosome 20q13.3 are identified herein as relevant biomarkers of PARP inhibitor sensitivity. As such, the presence of amplification of these biomarkers and/or chromosomal gains within these regions in the tumor tissues of patients can be used to identify individuals who are likely to benefit from PARP inhibitor therapies.

Accordingly, there are provided methods of identifying a subject having a poly-ADP ribose polymerase (PARP) inhibitor-sensitive tumor by detecting a genomic gain in chromosome 1q21 and/or chromosome 20q13.3 in a tumor sample from the subject, wherein the genomic gain is indicative of a tumor that is sensitive to PARP inhibitors.

In some embodiments, there are provided methods of identifying a subject having a poly-ADP ribose polymerase (PARP) inhibitor-sensitive tumor by detecting a genomic gain in chromosome 1q21 in a tumor sample from the subject, wherein the genomic gain is indicative of a tumor that is sensitive to PARP inhibitors.

In other embodiments, there are provided methods of treating a PARP inhibitor-sensitive tumor in a subject by detecting a genomic gain in chromosome 1q21 and/or chromosome 20q13.3 in a tumor sample from the subject, wherein the genomic gain is indicative of a tumor that is sensitive to PARP inhibitors, and administering an effective dose of a PARP inhibitor to the subject, thereby treating the PARP inhibitor-sensitive tumor.

In yet other embodiments, there are provided methods of treating a PARP inhibitor-sensitive tumor in a subject by detecting a genomic gain in chromosome 1q21 in a tumor sample from the subject, wherein the genomic gain is indicative of a tumor that is sensitive to PARP inhibitors, and administering an effective dose of a PARP inhibitor to the subject, thereby treating the PARP inhibitor-sensitive tumor.

In still other embodiments, there are provided method of treating a tumor with a genomic gain in chromosome 1q21 and/or chromosome 20q13.3 in a subject by administering an effective dose of a PARP inhibitor to a subject having a tumor with a genomic gain in chromosome 1q21 and/or chromosome 20q13.3, thereby treating the PARP inhibitor-sensitive tumor.

In other embodiments, there are provided method of treating a tumor with a genomic gain in chromosome 1q21 in a subject by administering an effective dose of a PARP inhibitor to a subject having a tumor with a genomic gain in chromosome 1q21, thereby treating the PARP inhibitor-sensitive tumor.

In particular aspects of the above methods, the genomic gain in chromosome 1q21 results in gene amplification of a CHD1L gene. In other aspects of the above methods, the genomic gain in chromosome 20q13.3 results in gene amplification of an RTEL1 gene.

### BRIEF DESCRIPTION OF THE DRAWINGS

FIG. 1 provides a box-and-whiskers plot for the 1q21 biomarker amplification status and relates the IC₅₀ distribution for the biomarker positive group of cell lines to the biomarker negative group of cell lines. The “box” is the interquartile range. The interface between the dark and light shading is the median IC₅₀. The top whisker extends to the maximum IC₅₀ value and the bottom whisker extends to the minimum IC₅₀ value. Please note in some instances the whiskers (vertical lines) exceed the graph.

FIG. 2 provides a box-and-whiskers plot for the 20q13 biomarker amplification status and relates the IC₅₀ distribution for the biomarker positive group of cell lines to the biomarker negative group of cell lines. The “box” is the interquartile range. The interface between the dark and light shading is the median IC₅₀. The top whisker extends to the maximum IC50 value and the bottom whisker extends to the minimum IC₅₀ value.

FIG. 3 is an image of a Western blot showing protein expression of CHD1L, tubulin, and β-actin in NSCLC and breast cancer cell lines.

FIG. 4 is an image of a Western blot showing protein expression of CHD1L and tubulin in 24 CHD1L knock-in clones and the parental cell line 184B5.

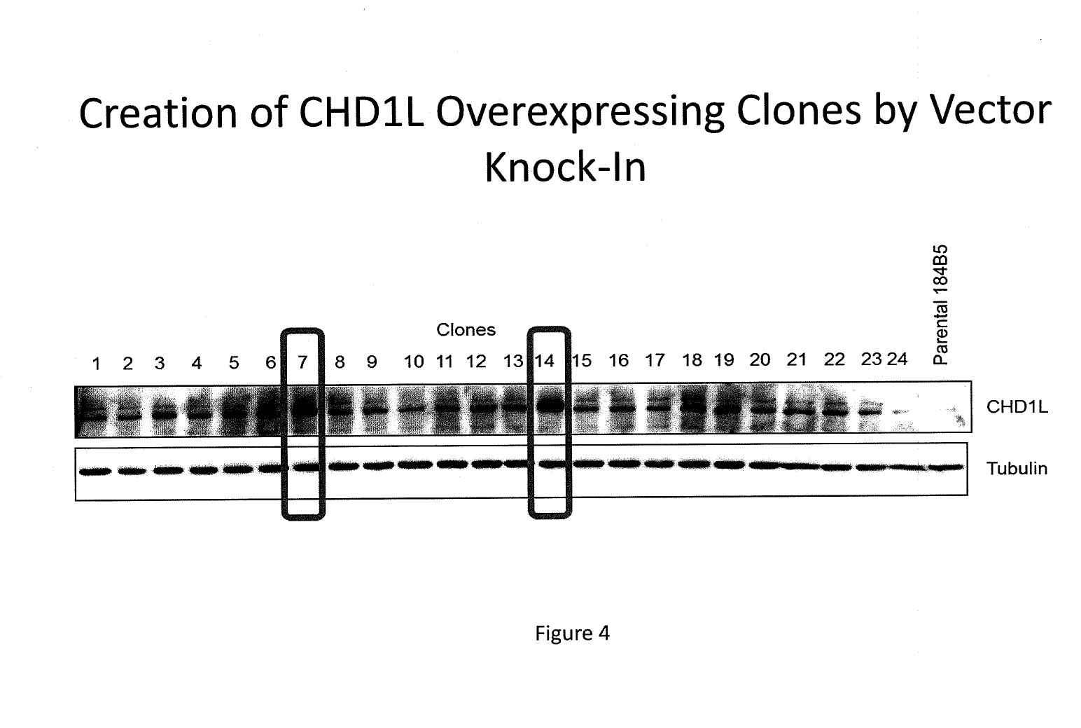

FIG. 5 shows plots of percent death from baseline (FIG. 5, top panel) and percent growth inhibition (FIG. 5, bottom panel) of two CHD1L knock-in clones (i.e., 184B5 clones 7 and 14) treated with BMN673, and compared to the parental cell line 184B5 treated with BMN673.

### DETAILED DESCRIPTION OF THE INVENTION

As provided herein, genomic gains in chromosomes 1q21 and 20q13.3 have been identified, which are strongly associated with predicting response of tumor cells to PARP inhibitors. In particular, gains of the gene CHD1L (also designated ALC1) located at chromosome 1q21 and/or the gene RTEL1 located at chromosome 20q13.3 are identified herein as relevant biomarkers of PARP inhibitor sensitivity. As such, the presence of amplification of these biomarkers and/or chromosomal gains within these regions in the tumor tissues of patients can be used to identify individuals who are likely to benefit from PARP inhibitor therapies. Amplification results in at least twice as many copies of the genes on the amplicon and gain implies low level (less than two fold) increases in the copy number.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of ±20% or ±10%, more preferably ±5%, even more preferably ±1% from the specified value, as such variations are appropriate to perform the disclosed methods.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “antibody” is meant to include intact molecules of polyclonal or monoclonal antibodies, chimeric, single chain, and humanized antibodies, as well as fragments thereof, such as Fab and F(ab′)₂, Fv and SCA fragments which are capable of binding an epitopic determinant. Monoclonal antibodies are made from antigen containing fragments of the protein by methods well known to those skilled in the art (Kohler, et al., Nature, 256:495, 1975). An Fab fragment consists of a monovalent antigen-binding fragment of an antibody molecule, and can be produced by digestion of a whole antibody molecule with the enzyme papain, to yield a fragment consisting of an intact light chain and a portion of a heavy chain. A Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An (Fab′)₂ fragment of an antibody can be obtained by treating a whole antibody molecule with the enzyme pepsin, without subsequent reduction. A (Fab′)₂ fragment is a dimer of two Fab′ fragments, held together by two disulfide bonds. An Fv fragment is defined as a genetically engineered fragment containing the variable region of a light chain and the variable region of a heavy chain expressed as two chains. A single chain antibody (“SCA”) is a genetically engineered single chain molecule containing the variable region of a light chain and the variable region of a heavy chain, linked by a suitable, flexible polypeptide linker.

“Nucleic acid” or “oligonucleotide” or “polynucleotide” or grammatical equivalents used herein means at least two nucleotides covalently linked together. Nucleic acids are typically deoxyribonucleotide or ribonucleotides polymers (pure or mixed) in single- or double-stranded form. The term may encompass nucleic acids containing nucleotide analogs or modified backbone residues or linkages, which are synthetic, naturally occurring, and non-naturally occurring, which have similar binding, structural, or functional properties as the reference nucleic acid, and which are metabolized in a manner similar to the reference nucleotides. Non-limiting examples of such analogs include, without limitation, phosphorothioates, phosphoramidates, methyl phosphonates, chiral-methyl phosphonates, 2-O-methyl ribonucleotides, and peptide-nucleic acids (PNAs). A nucleic acid will generally contain phosphodiester bonds, although in some cases, nucleic acid analogs are included that may have at least one different linkage, e.g., phosphoramidate, phosphorothioate, phosphorodithioate, or O-methylphosphoroamidite linkages. The term nucleic acid may, in some contexts, be used interchangeably with gene, cDNA, mRNA, oligonucleotide, and polynucleotide.

A particular nucleic acid sequence also encompasses conservatively modified variants thereof (such as degenerate codon substitutions) and complementary sequences, as well as the sequence explicitly indicated. Specifically, degenerate codon substitutions may be achieved by generating sequences in which the third (“wobble”) position of one or more selected (or all) codons is substituted with mixed-base and/or deoxyinosine residues. Thus a nucleic acid sequence encoding a protein sequence disclosed herein also encompasses modified variants thereof as described herein.

The terms “polypeptide”, “peptide”, and “protein” are typically used interchangeably herein to refer to a polymer of amino acid residues. Amino acids may be referred to herein by either their commonly known three letter symbols or by the one-letter symbols recommended by the IUPAC-IUB Biochemical Nomenclature Commission.

As used herein, the terms “sample” and “biological sample” refer to any sample suitable for the methods provided by the present invention. In preferred embodiments, the sample contains nucleic acid and/or protein. In one embodiment, the biological sample of the present invention is a tissue sample, e.g., a biopsy specimen such as samples from needle biopsy, core needle biopsy or excisional biopsy (i.e., biopsy sample). In other embodiments, the biological sample of the present invention is a sample of bodily fluid, e.g., blood, serum, plasma, sputum, lung aspirate, or urine.

As used herein, the term “amplification” when used in reference to a gene or amplicon means a log 2(ratio)>1, in other words, the amplification event results in at least twice as many copies of the gene or the amplicon. As used herein, the term “gain” typically refers to a low level increase in copy number (i.e., less than a 2-fold increase).

Reference herein to “normal cells” or “corresponding normal cells” means cells that are from the same organ and of the same type as the cancer cell type. In one aspect, the corresponding normal cells comprise a sample of cells obtained from a healthy individual. Such corresponding normal cells can, but need not be, from an individual that is age-matched and/or of the same gender as the individual providing the cancer cells being examined. In another aspect, the corresponding normal cells comprise a sample of cells obtained from an otherwise healthy portion of tissue of a subject having cancer. In some embodiments of the present methods, the determination of a genomic gain is made by comparison of the genome from a cancer or tumor sample to a normal cell.

A cell line panel consisting of more than 600 human cancer cell lines originally derived from actual individual patient malignancies representing a broad spectrum of common human cancers, including 15 separate histologic subtypes, e.g. breast, ovary, lung, colorectal, gastric, melanoma, pancreas, etc. has been collected and comprehensively characterized. Specifically, this panel has been characterized with regard to the individual cell line's ability to grow in vitro on plastic and in soft agar, as well as in vivo growth subcutaneously and ortho-topically. In addition, each cell line in the panel has been characterized for gene expression by transcript microarray, as well as gene copy number variation (CNV). Using this characterized panel, preclinical, growth inhibition studies with various new potential therapeutics or novel combinations of therapeutics may be performed to determine which lines and/or histologies do or do not respond to the therapeutic intervention being assessed.

Using this research platform, the present inventors have completed studies with inhibitors of poly-ADP ribose polymerase (PARP). PARP is an enzyme that plays a critical role in DNA repair and recently, alterations or changes in DNA repair pathways have been implicated in the pathogenesis of some human cancers. Consequently, PARP inhibition has been put forward as a potential strategy to treat human cancers. Several small molecule inhibitors of PARP activity have been developed and brought forward into clinical development. Some have shown growth inhibitory activity in a small but distinct number of human cancer cell lines and patient tumors that lack specific DNA repair mechanisms either through inherited mutations and/or non-inherited silencing of genes like BRCA-1 and 2. Other known genes encoding proteins critical to DNA repair functions have also been implicated as mutation targets in the malignant process of some cancers.

As provided herein, however, growth inhibitory activity of PARP inhibitors has been observed using the above cell line panel, in cell lines other than those that contain mutations in BRCA-1, BRCA-2, or other DNA repair genes. Taken together these data indicate a potential therapeutic role for PARP inhibitors beyond those cancers containing alterations in the known or usual DNA repair genes commonly suspected of playing a role in cancer pathogenesis. By extension, these data further indicate that there are other alterations that might identify PARP-responsive human cancers, opening the possibility that we could use our large, biologically and molecularly characterized human cancer cell line panel to identify such predictive biomarkers. Indeed, as provided herein, it has been determined that genomic gains in chromosomes 1q21 and 20q13.3 are strongly associated with predicting response to PARP inhibitors, in experiments using our panel of molecularly characterized human cancer cell lines. More specifically, it is believed that the gains are of the gene CHD1L (also designated ALC1) located at chromosome 1q21 and/or the gene RTEL1 located at chromosome 20q13.3, and thus these genes are biomarkers of PARP inhibitor sensitivity. As such, the presence of gene amplification and/or chromosomal gains of these biomarkers in the malignant tissues of patients can be used to identify individuals who are likely to benefit from PARP inhibitor therapies.

In some embodiments, there are provided methods of identifying a subject having a poly-ADP ribose polymerase (PARP) inhibitor-sensitive tumor by detecting a genomic gain in chromosome 1q21 and/or chromosome 20q13.3 in a tumor sample from the subject, wherein the genomic gain is indicative of a tumor that is sensitive to PARP inhibitors. In particular aspects of the above methods, the genomic gain in chromosome 1q21 results in gene amplification of a CHD1L gene. In other aspects of the above methods, the genomic gain in chromosome 20q13.3 results in gene amplification of an RTEL1 gene.

In some embodiments, the alteration (e.g., chromosomal gain or gene amplification) from normal of two different biomarkers of response to PARP inhibition in human cancers is measured. These biomarker alterations are the result of gains at two chromosomal loci; 1q21 and 20_{q}13.3. The two genes that are believed to be the responsible for conferring sensitivity to PARP inhibition are CHD1L at 1q21 and RTEL1 at 20q13.3, respectively.

Detection of alterations at the DNA level can be by techniques well-known in the art to detect increases in DNA copy number at the 1q21 and 20q13.3 loci. In some embodiments, genomic gain may be detected using techniques such as (but not limited to) single nucleotide polymorphism (SNP) arrays, comparative genomic hybridization (CGH), Southern blot analysis or florescent in situ hybridization (FISH).

Methods of evaluating the presence and/or copy number of a particular gene are well known to those of skill in the art. For example, hybridization based assays can be used for these purposes.

Hybridization assays can be used to detect copy number of CHD1L and/or RTEL1. Hybridization-based assays include, but are not limited to, traditional “direct probe” methods such as Southern blots or in situ hybridization (e.g., fluorescence in situ hybridization (FISH)), and “comparative probe” methods such as comparative genomic hybridization (CGH). The methods can be used in a wide variety of formats including, but not limited to substrate-bound (e.g. membrane or glass) methods or array-based approaches as described below.

In a typical in situ hybridization assay, cells or tissue sections are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes are typically labeled, e.g., with radioisotopes or fluorescent reporters. Preferred probes are sufficiently long so as to specifically hybridize with the target nucleic acid(s) under stringent conditions. The preferred size range is from about 200 bp to about 1000 bases.

In some applications it is necessary to block the hybridization capacity of repetitive sequences. Thus, in some embodiments, tRNA, human genomic DNA, or Cot-1 DNA is used to block non-specific hybridization.

In comparative genomic hybridization methods a first collection of (sample) nucleic acids (e.g. from a tumor) is labeled with a first label, while a second collection of (control) nucleic acids (e.g. from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), etc. In one particularly preferred embodiment, the hybridization protocol of Pinkel et al. (1998) Nature Genetics 20: 207-211, or of Kallioniemi (1992) Proc. Natl Acad Sci USA 89:5321-5325 (1992) is used.

A variety of nucleic acid hybridization formats are known to those skilled in the art. For example, common formats include sandwich assays and competition or displacement assays. Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

Typically, labeled signal nucleic acids are used to detect hybridization. The labels may be incorporated by any of a number of means well known to those of skill in the art. Means of attaching labels to nucleic acids include, for example nick translation, or end-labeling by kinasing of the nucleic acid and subsequent attachment (ligation) of a linker joining the sample nucleic acid to a label (e.g., a fluorophore). A wide variety of linkers for the attachment of labels to nucleic acids are also known. In addition, intercalating dyes and fluorescent nucleotides can also be used.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., DYNABEADS), fluorescent labels (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277, U.S. Pat. No. 437; 4,275,149; and U.S. Pat. No. 4,366,241.

The label may be added to the nucleic acids prior to, or after the hybridization. So called “direct labels” are detectable labels that are directly attached to or incorporated into the sample or probe nucleic acids prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y., (1993)).

The methods of this invention may be performed with array-based hybridization formats. For a description of one preferred array-based hybridization system see Pinkel et al. (1998) Nature Genetics, 20: 207-211.

Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211). Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

The DNA used to prepare the arrays of the invention is not critical. For example the arrays can include genomic DNA, e.g. overlapping clones that provide a high resolution scan of a portion of the genome containing the desired gene, or of the gene itself. Genomic nucleic acids can be obtained from, e.g., HACs, MACs, YACs, BACs, PACs, P1s, cosmids, plasmids, inter-Alu PCR products of genomic clones, restriction digests of genomic clones, cDNA clones, amplification (e.g., PCR) products, and the like.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays.

In other embodiments, amplification-based assays can be used to measure CHD1L and/or RTEL1 gene copy number in a sample. In such amplification-based assays, the nucleic acid sequences act as a template in an amplification reaction (e.g. Polymerase Chain Reaction (PCR)). In a quantitative amplification, the amount of amplification product will be proportional to the amount of template in the original sample. Comparison to appropriate (e.g. healthy tissue) controls provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). The known nucleic acid sequence for the genes is sufficient to enable one of skill to routinely select primers to amplify any portion of the gene.

Real time PCR is another amplification technique that can be used to determine gene copy levels or levels of mRNA expression. (See, e.g., Gibson et al., Genome Research 6:995-1001, 1996; Heid et al., Genome Research 6:986-994, 1996). Real-time PCR is a technique that evaluates the level of PCR product accumulation during amplification. This technique permits quantitative evaluation of mRNA levels in multiple samples. For gene copy levels, total genomic DNA is isolated from a sample. For mRNA levels, mRNA is extracted from tumor and normal tissue and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, β-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Other suitable amplification methods include, but are not limited to ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Because these DNA-based chromosomal gains/gene amplification are associated with overexpression of the respective gene products, the alteration can be measured in cancer cells or tumor tissue using any technique that determines chromosomal gains/gene amplification at the DNA level, as well as gene expression at the RNA or protein levels.

To detect the alterations at the RNA level, techniques such as (but not be limited to) transcript expression arrays, RNA in situ hybridization, northern blot analysis, transcript enumeration via direct exon/transcript sequencing (e.g. Lumina sequencing platforms) may be employed to detect increases in mRNA gene expression of the CHD1L or RTEL1 transcripts. To detect the alterations at the protein level, this would include (but not be limited to) techniques such as protein arrays (e.g. reverse phase protein analysis-RPPA) or western blot analysis of cell or tissue lysates/extracts, immunohistochemical staining analysis of tissue sections for the present of the CHD1 L or RTEL1 target proteins, or any antibody-based methodology directed at detecting increases in protein expression of the target proteins, CHD1L or RTEL1. Thus, in some embodiments, the gene expression is measured using transcript expression array analysis, RNA in situ hybridization, northern blot analysis, transcript enumeration by direct exon/transcript sequencing, protein array analysis, western blot analysis, immunohistochemical tissue staining, or immunoassay. In some aspects, gene amplification is detected by measuring an increase in gene expression of CHD1L and/or RTEL1 as compared to gene expression of CHD1L and/or RTEL1 in normal cells from the subject.

CHD1L or RTEL1 gene expression level can also be assayed as a marker for cancer. In preferred embodiments, activity of the CHD1L or RTEL1 gene is determined through a measure of gene transcript (e.g. mRNA), by a measure of the quantity of translated protein, or by a measure of gene product activity.

Methods of detecting and/or quantifying the gene transcript (mRNA or cDNA) using nucleic acid hybridization techniques are known to those of skill in the art (see Sambrook et al. Molecular Cloning, A Laboratory Manual, Cold Spring Harbor Press, 1989).

For example, one method for evaluating the presence, absence, or quantity of mRNA involves a Northern blot transfer. The probes for use in Northern blotting can be full length or less than the full length of the nucleic acid sequence encoding the CHD1L or RTEL1 protein. Shorter probes are empirically tested for specificity. Preferably nucleic acid probes are 20 bases or longer in length. (See Sambrook et al., supra, for methods of selecting nucleic acid probe sequences for use in nucleic acid hybridization.) Visualization of the hybridized portions allows the qualitative determination of the presence or absence of mRNA.

In another example, a CHD1L or RTEL1 transcript (e.g., mRNA) can be measured using amplification (e.g. PCR) based methods as described above for directly assessing copy number of DNA. In a preferred embodiment, transcript level is assessed by using reverse transcription PCR (RT-PCR). In another preferred embodiment, transcript level is assessed using real-time PCR.

The expression level of a CHD1L or RTEL1 gene can also be detected and/or quantified by detecting or quantifying the expressed CHD1L or RTEL1 polypeptide. The polypeptide can be detected and quantified by any of a number of means well-known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

Immunohistochemical methods can also be used to detect CHD1L or RTEL1 protein. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu et al. (1980) Am. J. Clin. Path. 75:734-738. The isolated proteins can also be sequenced according to standard techniques to identify polymorphisms.

The CHD1L or RTEL1 polypeptide can be detected and/or quantified using any of a number of well-known immunological binding assays (see, e.g., U.S. Pat. No. 4,366,241; U.S. Pat. No. 4,376,110; U.S. Pat. No. 4,517,288; and U.S. Pat. No. 4,837,168). For a review of the general immunoassays, see also Asai (1993) Methods in Cell Biology Volume 37: Antibodies in Cell Biology, Academic Press, Inc. New York; Stites & Ten (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled anti-antibody. Alternatively, the labeling agent may be a third moiety, such as another antibody, that specifically binds to the antibody/polypeptide complex.

In one preferred embodiment, the labeling agent is a second human antibody bearing a label. Alternatively, the second antibody may lack a label, but it may, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin. In some embodiments, Western blot analysis is used to detected and or quantify CHD1L or RTEL1 protein.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

CHD1L or RTEL1 protein can be detected and/or quantified in cells using immunocytochemical or immunohistochemical methods. IHC (immunohistochemistry) can be performed on paraffin-embedded tumor blocks using a CHD1L or RTEL1-specific antibody. IHC is the method of colormetric or fluorescent detection of archival samples, usually paraffin-embedded, using an antibody that is placed directly on slides cut from the paraffin block. To detect and/or quantify CHD1L or RTEL1 in, for example tissue culture cells or cells from a subject that are not embedded in paraffin (for example, hematopoetic cells) ICC (immunocytochemistry) can be used. ICC is like IHC but uses fresh, non-paraffin embedded cells plated onto slides and then fixed and stained.

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of peptides can be determined by conventional techniques to determine the magnitude of the antibody response of an animal that has been immunized with the peptide. Generally, the peptides that are used to raise the anti-peptide antibodies should generally be those which induce production of high titers of antibody with relatively high affinity for the polypeptide.

Preferably, the antibodies produced will be monoclonal antibodies (“mAbs”). For preparation of monoclonal antibodies, immunization of a mouse or rat is preferred. Polyclonal antibodies can also be used.

The assays of this invention have immediate utility in detecting/predicting the likelihood of a cancer, in estimating survival from a cancer, in screening for agents that modulate the subject gene product activity, and in screening for agents that inhibit cell proliferation.

In some embodiments, malignant tissue specimens of cancers from individual patients may be tested for the presence of alterations in the 1q21 or 20q13.3 loci and/or the genes CHD1L or RTEL1 genes by any of the methods provided herein. If the types of alterations listed in this disclosure are found to be present, these patients would be considered as appropriate candidates to receive PARP inhibitor-based therapies as part of the treatment regimen for their cancers.

In other embodiments, there are provided methods of treating a PARP inhibitor-sensitive tumor in a subject by detecting a genomic gain in chromosome 1q21 and/or chromosome 20q13.3 in a tumor sample from the subject, wherein the genomic gain is indicative of a tumor that is sensitive to PARP inhibitors, and administering an effective dose of a PARP inhibitor to the subject, thereby treating the PARP inhibitor-sensitive tumor.

In still other embodiments, there are provided method of treating a tumor with a genomic gain in chromosome 1q21 and/or chromosome 20q13.3 in a subject by administering an effective dose of a PARP inhibitor to a subject having a tumor with a genomic gain in chromosome 1q21 and/or chromosome 20q13.3, thereby treating the PARP inhibitor-sensitive tumor.

As used herein, “treatment” or “treating” is an approach for obtaining beneficial or desired clinical results. For purposes of this invention, beneficial or desired clinical results include, but are not limited to, reduction in size or elimination of cancer or pre-cancerous tumors, inhibition or reduction in cancer cell growth, and/or induction of cancer cell death.

The terms “tumor,” “cancer,” and “neoplasm,” are used interchangeably herein to refer to cells which exhibit autonomous, unregulated growth, such that they exhibit an aberrant growth phenotype characterized by a significant loss of control over cell proliferation. In general, cells of interest for detection, analysis, classification, or treatment in the present invention include precancerous (e.g., benign), malignant, pre-metastatic, metastatic, and non-metastatic cells. Examples of cancer include but are not limited to, breast cancer, colon cancer, cervical cancer, ovarian cancer, lung cancer, prostate cancer, testicular cancer, bladder cancer, cancer of the urinary tract, hepatocellular cancer, gastric cancer, stomach cancer, pancreatic cancer, liver cancer, thyroid cancer, renal cancer, carcinoma, melanoma, head and neck cancer, leukemia, lymphoma, and brain cancer.

Thus, the tumor treated by with the invention methods may be any cancer or precancerous tumor sensitive to PARP inhibition. In particular embodiments, the PARP inhibitor-sensitive tumor is cancer. In some embodiments, the cancer is breast cancer, colon cancer, cervical cancer, ovarian cancer, lung cancer, prostate cancer, testicular cancer, bladder cancer, cancer of the urinary tract, hepatocellular cancer, gastric cancer, stomach cancer, pancreatic cancer, liver cancer, thyroid cancer, renal cancer, carcinoma, melanoma, head and neck cancer, leukemia, lymphoma, or brain cancer. In certain embodiments, the cancer is a breast cancer, an ovarian cancer, a lung cancer, a bladder cancer, a liver cancer, a head and neck cancer, or a colorectal cancer. In some aspects, the cancer is breast cancer or ovarian cancer. In other aspects, the cancer lacks a BRCA-1 or BRCA-2 mutation.

The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration or administration via intranasal delivery. In the present methods of treatment, the PARP inhibitor may be administered by any of these routes.

In some embodiments, the PARP inhibitor is administered as a composition including a pharmaceutically acceptable carrier or vehicle. The term “pharmaceutically acceptable,” when used in reference to a carrier, is meant that the carrier, diluent or excipient must be compatible with the other ingredients of the formulation and not deleterious to the recipient thereof. Such a component is one that is suitable for use with humans, animals, and/or plants without undue adverse side effects. Non-limiting examples of adverse side effects include toxicity, irritation, and/or allergic response. The term “carrier” refers to a diluent, adjuvant, excipient, or vehicle with which the PARP inhibitor is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water can be a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the PARP inhibitor, preferably in purified form, together with a suitable amount of carrier so as to provide the form for proper administration to the patient. The formulation should suit the mode of administration.

In a specific embodiment, the composition is formulated, in accordance with routine procedures, as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

In some embodiments, PARP inhibitor may be olaparib, isoindolinone derivatives, veliparib, iniparib, BMN673, or 4-methoxy-carbazole derivatives. In particular embodiments, the PARP inhibitor is ABT472, ABT767, ABT888 (veliparib), AZD2281 (olaparib), AZD2461, BeiGene290, BMN673, BSI101, BSI201 (iniparib), BSI401, CEP8983, CEP9722, CO338 (rucaparib phosphate), CPH101 with CPH102, E7016, E7449, IMP04149, IMP4297, INO1001, INO1003, JPI283, JPI289, KU0687, MK4827 (niraparib), NT125, or SOMCL9112. The PARP inhibitor may be administered as the sole therapeutic agent (i.e., monotherapy) or may be administered in conjunction with another therapeutic agent. Such other therapeutic agents may be antineoplastic agents. The PARP inhibitor and antineoplastic agents can be administered simultaneously or sequentially by the same or different routes of administration. The determination of which antineoplastic agent(s) and amount to use in combination with PARP inhibitors in a method of the present invention can be readily made by ordinarily skilled medical practitioners using standard techniques known in the art, and will vary depending on the type and severity of cancer being treated.

As used herein, an “effective amount” is an amount of a substance or composition sufficient to effect beneficial or desired clinical results including inhibition or reduction in cancer cell growth and/or induction of cancer cell death (i.e., apoptosis). For purposes of this invention, an effective amount of a PARP inhibitor is an amount sufficient to reduce cancer cell growth. In some embodiments, the “effective amount” may be administered before, during, and/or after any treatment regimens for cancer.

The total amount of a compound or composition to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of each component of the synergistic composition used to treat cancer in a subject depends on many factors including the age and general health of the subject as well as the route of administration and the number of treatments to be administered. In view of these factors, the skilled artisan would adjust the particular dose as necessary.

The following examples are intended to illustrate, but not limit the invention.

### Example 1

This example illustrates the identification of biomarkers for tumors sensitive to PARP inhibition. Specifically, two biomarkers that are associated with a two-to-three fold increase in response ratios to PARP inhibition were identified by interrogating 332 human cancer cell lines. These biomarkers were located in two areas of chromosomal gain. One gain occurred in the region of the CHD1L gene on chromosome 1q21 and was found in 32 of 346 cell lines. Table 1 below shows the number of cell lines according to the tissue of the primary tumor from which it was derived that exhibited the 1q21 chromosomal gain. (The amplifications were detected by Comparative Genomic Hybridization (CGH) array. Gene location was determined from the CGH analytical software.) This alteration carried a PARP inhibitor response ratio of 2.76 (95% CI=1.77-4.31; p<0.0001). The second area of gain was in the region of the RTEL1 gene on chromosome 20q13.3 and was found in 47 of 346 cell lines. (See Table 1 below for the number of cell lines exhibiting the 20q13.3 chromosomal gain.) This alteration carried a PARP inhibitor response ratio of 2.27 (95% CI=1.45-3.57; p=0.0008). Overall, cell lines containing either alteration (69/346) had a response ratio of 2.62 (95% CI=1.73-3.96; p<0.0001) while those that contained both biomarkers (10/346) had a response ratio of 3.22 (95% CI=1.84-5.61; p=0.001).

TABLE 1

| Primary | Total Cell | 1q21 | 20q13 | | |
|---|---|---|---|---|---|
| Tumor Tissue | Lines | Amps | Amps | Either | Both |
| Bladder | 27 | 2 | 4 | 5 | 1 |
| Breast | 45 | 9 | 7 | 14 | 2 |
| Colon | 23 | 1 | 5 | 6 | 0 |
| Endometrial | 18 | 1 | 2 | 2 | 1 |
| Head and | 30 | 3 | 1 | 4 | 0 |
| Neck | | | | | |
| Liver | 20 | 0 | 1 | 1 | 0 |
| Lung | 55 | 11 | 10 | 17 | 4 |
| Melanoma | 46 | 1 | 7 | 8 | 0 |
| Ovarian | 39 | 4 | 6 | 8 | 2 |
| Pancreas | 29 | 0 | 1 | 1 | 0 |
| Upper GI | 14 | 0 | 3 | 3 | 0 |
| TOTAL: | 346 | 32 | 47 | 69 | 10 |

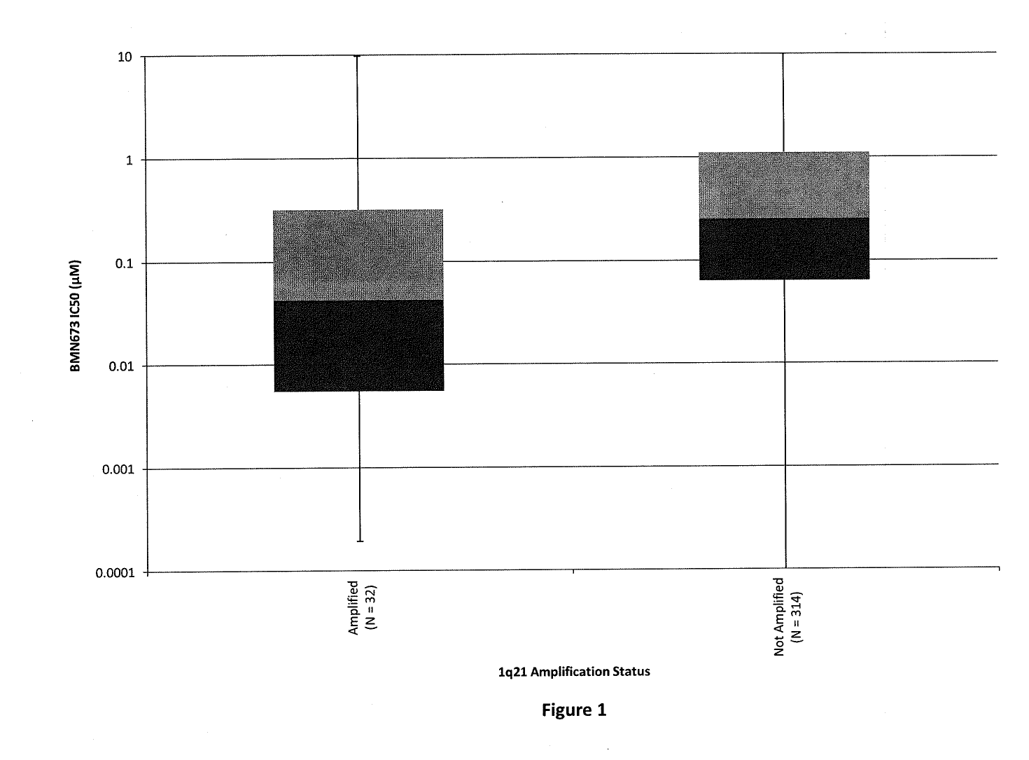

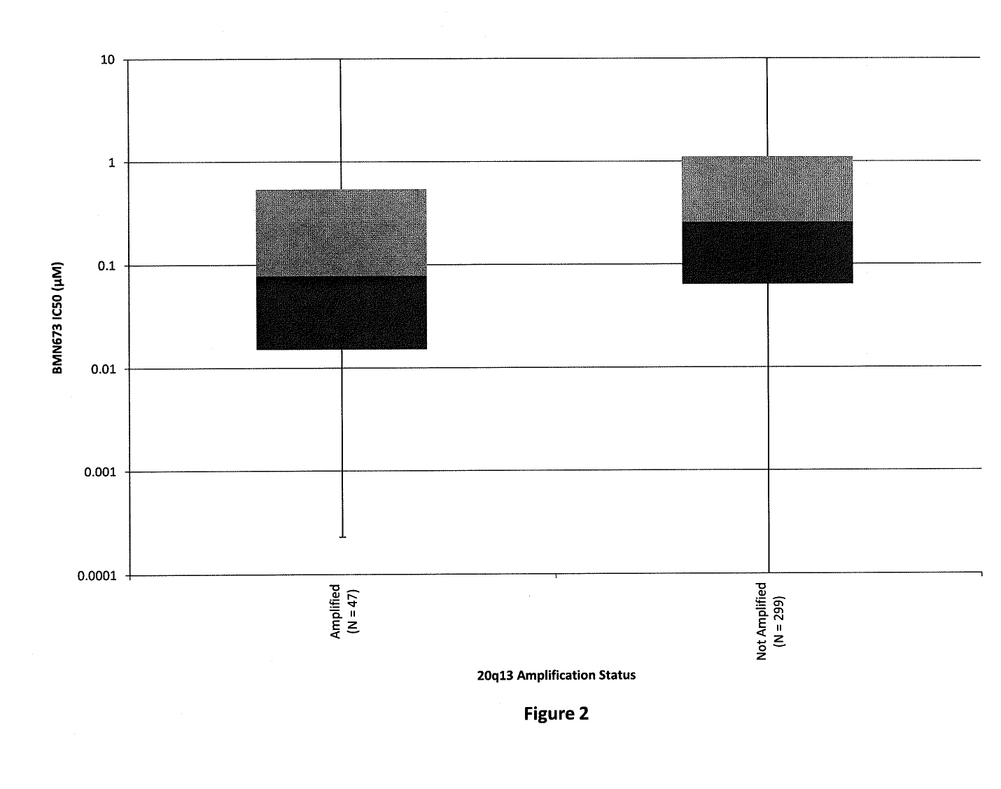

FIGS. 1 and 2 provide box-and-whiskers plots for the respective biomarkers, 1q21 and 20q13, and show the IC₅₀ distribution for the amplified biomarker positive group of cell lines versus the not amplified biomarker negative group of cell lines. The “box” is the interquartile range. The interface between the dark and light shading within the box is the median IC₅₀. The top whisker extends to the maximum IC₅₀ value and the bottom whisker extends to the minimum IC₅₀ value.

### Example 2

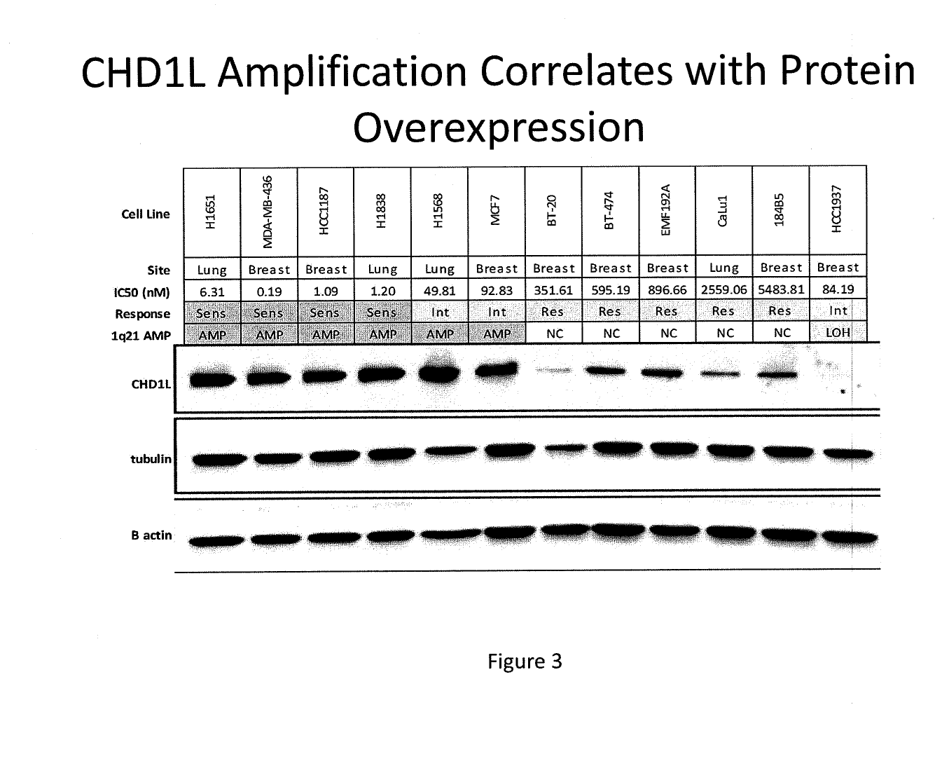

This example illustrates that CHD1L amplification correlated with overexpression of CHD1L protein. Non-small cell lung cancer (NSCLC) and breast cancer cell lines were assayed for sensitivity to the PARP inhibitor BMN673, and for CHD1L expression and were found to have different BMN673 sensitivities, CHD1L protein expression, and CHD1L genomic characteristics. CHD1L protein expression in the NSCLC and breast cancer cell lines was assayed by Western blot with antibodies against CHD1L. High expression of CHD1L protein was observed in the following cell lines: H1651, MDA-MB-436, HCC1187, H1838, and MCF7 (FIG. 3), whereas very little CHD1L protein expression was observed in the following cell lines: BT-20, BT-474, EMF-192A, Calu1, and 184B5 (FIG. 3). HCC1937 was used as a negative control for CHD1L protein expression, as the cell line carries a loss of heterozygosity (LOH) of CHD1L.

Each of these cell lines was also assayed for sensitivity or resistance to inhibition of PARP by BMN673. Briefly, each cell line was cultured and exposed to a range of concentrations of BMN673 to determine an IC₅₀ value of inhibitory effect on PARP (as evidenced by cell growth) for each cell line (see Table 2). BMN673 sensitive cell lines were found to be H1651, MDA-MB-436, HCC1187, H1838 and MCF7. BMN673 resistant cell lines were found to be BT-20, BT-474, EMF-192A, Calu1, and 184B5.

Each of the above NSCLC and breast cancer cell lines cell lines were also assayed for amplification of chromosome 1q21. The amplifications were detected by Comparative Genomic Hybridization (CGH) array. Cell lines that exhibited amplification of chromosome 1q21 (designated by “AMP” in Table 2) were H1651, MDA-MB-436, HCC1187, H1838 and MCF7. Cell lines that did not exhibit amplification of chromosome 1q21 (designated by “NC” in Table 2) were BT-20, BT-474, EMF-192A, Calu1, and 184B5.

Each of the sensitive cell lines showed genomic amplification for CHD1L, which correlated with high CHD1L protein expression. Each of the resistant cell lines did not show chromosome 1q21 amplification, and showed very little CHD1L protein expression by Western blot.

TABLE 2

| Cell Line | H1651 | MDA-MB-436 | HCC1187 | H1838 | H1568 | MCF7 | BT-20 | BT-474 | EMF192A | CaLu1 | 184B5 | HCC1937 |
|---|---|---|---|---|---|---|---|---|---|---|---|---|
| Site | Lung | Breast | Breast | Lung | Lung | Breast | Breast | Breast | Breast | Lung | Breast | Breast |
| IC50 (nM) | 6.31 | 0.19 | 1.09 | 1.20 | 49.81 | 92.83 | 351.61 | 595.19 | 896.66 | 2559.06 | 5483.81 | 84.19 |
| Response | Sens | Sens | Sens | Sens | Int | Int | Res | Res | Res | Res | Res | Int |
| 1q21 AMP | AMP | AMP | AMP | AMP | AMP | AMP | NC | NC | NC | NC | NC | LOH |

### Example 3

This example illustrates that CHD1L amplification is detectable by fluorescent in situ hybridization (FISH) in MCF7 cells. FISH analysis was performed on two cell lines with a probe for CHD1L DNA. A wild type cell line (184B5) showed two copies of CHD1L in G0/G1 cells. A CHD1L-amplified line (MCF7) showed 5-6 copies of CHD1L.

### Example 4

This example illustrates the creation of cell lines overexpressing CHD1L protein by vector knock-in. Briefly, breast cancer cell line 184B5 cells were transduced with lentivirus containing CHD1L cDNA. After transduction, clones transfected with CHD1L were selected with 500 μg/mL G418 for 1 week. Expression of CHD1L protein in the 24 knock-in clones and the parental cell line 184B5 was detected by Western blot. Clone 7 and clone 14 exhibited high CHD1L protein expression as compared with the parental cell line and were used in the subsequent BMN673 sensitivity study.

### Example 5

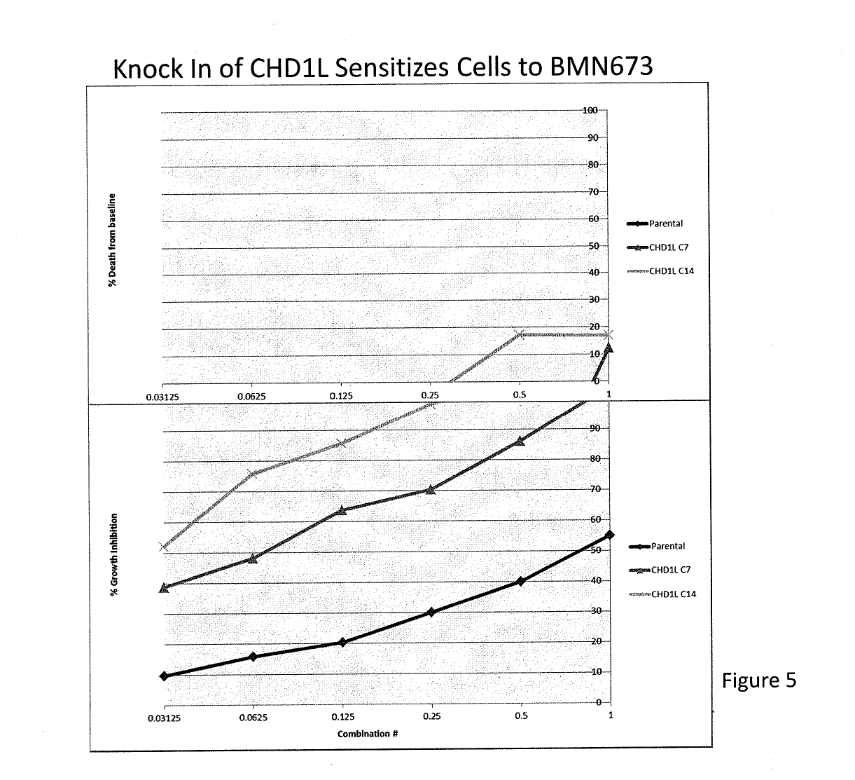

This example illustrates that the knock-in of CHD1L protein sensitized breast cancer cells to BMN673 treatment. Briefly, 184B5 knock-in clones 7 and 14 (i.e., cells with high CHD1L protein expression) were treated with BMN673 and compared with the 184B5 parental cell line (i.e., cells with low CHD1L protein expression) treated with BMN673, with respect to percent death from baseline (FIG. 5, top panel) and percent growth inhibition (FIG. 5, bottom panel). A shift in BMN673 IC₅₀ from ˜1 micromolar (IC₅₀ of the BMN673 resistant parental cell line 184B5) to 62 nanomolar (clone 7) and 30 nanomolar (clone 14) demonstrated that the knock-in cell lines (i.e., clone 7 and clone 14) were converted to BMN673 sensitive cell lines.

Although the invention has been described with reference to the above example, it will be understood that modifications and variations are encompassed within the spirit and scope of the invention. Accordingly, the invention is limited only by the following claims.

What is claimed is:

1. A method of identifying a subject having a poly-ADP ribose polymerase (PARP) inhibitor-sensitive tumor, comprising,
detecting a genomic gain in chromosome 1q21 and/or chromosome 20q13.3 in a tumor sample from the subject, wherein the genomic gain is indicative of a tumor that is sensitive to PARP inhibitors.

2. A method of treating a PARP inhibitor-sensitive tumor in a subject comprising,
detecting a genomic gain in chromosome 1q21 and/or chromosome 20q13.3 in a tumor sample from the subject, wherein the genomic gain is indicative of a tumor that is sensitive to PARP inhibitors, and
administering an effective dose of a PARP inhibitor to the subject, thereby treating the PARP inhibitor-sensitive tumor.

3. A method of treating a tumor with a genomic gain in chromosome 1q21 and/or chromosome 20q13.3 in a subject comprising,
administering an effective dose of a PARP inhibitor to a subject having a tumor with a genomic gain in chromosome 1q21 and/or chromosome 20q13.3, thereby treating the PARP inhibitor-sensitive tumor.

4. The method of any one of claim 1, 2, or 3, wherein the genomic gain is detected using a single nucleotide polymorphism (SNP) array, comparative genomic hybridization (CGH), southern blot analysis, or fluorescent in situ hybridization (FISH).

5. The method of claim 4, wherein the genomic gain is determined by comparison to a genome of a normal cell.

6. The method of any one of claim 1, 2, or 3, wherein the genomic gain in chromosome 1q21 results in gene amplification of a CHD1L gene.

7. The method of any one of claim 1, 2, or 3, wherein the genomic gain in chromosome 20q13.3 results in gene amplification of an RTEL1 gene.

8. The method of claim 6 or claim 7, wherein the gene amplification is detected by measuring an increase in gene expression of CHD1L and/or RTEL1 as compared to gene expression of CHD1L and/or RTEL1 in normal cells from the subject.

9. The method of claim 8, wherein gene expression is measured using transcript expression array analysis, RNA in situ hybridization, northern blot analysis, transcript enumeration by direct exon/transcript sequencing, protein array analysis, western blot analysis, immunohistochemical tissue staining, or immunoassay.

10. The method of claim 7, wherein the gene amplification is detected by measuring an increase in gene expression of CHD1L and/or RTEL1 as compared to gene expression of CHD1L and/or RTEL1 in normal cells from the subject.

11. The method of claim 10, wherein gene expression is measured using transcript expression array analysis, RNA in situ hybridization, northern blot analysis, transcript enumeration by direct exon/transcript sequencing, protein array analysis, western blot analysis, immunohistochemical tissue staining, or immunoassay.

12. The method of any one of claim 1, 2, or 3, wherein the tumor is selected from the group consisting of a breast cancer, an ovarian cancer, a lung cancer, a bladder cancer, a liver cancer, a head and neck cancer, a pancreatic cancer, a gastrointestinal cancer, and a colorectal cancer.

13. The method of claim 2 or claim 3, wherein the PARP inhibitor is selected from the group consisting of isoindolinone derivatives, ABT472, ABT767, ABT888 (veliparib), AZD2281 (olaparib), AZD2461, BeiGene290, BMN673, BSI101, BSI201 (iniparib), BSI401, CEP8983, CEP9722, CO338 (rucaparib phosphate), CPH101 with CPH102, E7016, E7449, IMP04149, IMP4297, INO1001, INO1003, JPI283, JPI289, KU0687, MK4827 (niraparib), NT125, SOMCL9112, and 4-methoxy-carbazole derivatives.

14. The method of claim 2 or claim 3, further comprising administering an effective dose of a further therapeutic agent.

15. The method of claim 14, wherein the further therapeutic agent is an antineoplastic agent.
